(19) United States
(12) Patent Application Publication
(10) Pub. No.: US 2021/0138203 A1
(43) Pub. Date: May 13, 2021

(54) HELICAL GUIDEWIRES AND RELATED SYSTEMS

(71) Applicants: Robert Lederman, Chevy Chase, MD (US); Adam Greenbaum, Andover, MA (US); Nasser Rafiee, Andover, MA (US); Stuart MacDonald, andover, MA (US); Mai Le Diep, andover, MA (US); Rogers Toby, andover, MA (US); Jaffar Khan, Andover, MA (US); John Horrigan, Andover, MA (US)

(72) Inventors: Robert Lederman, Chevy Chase, MD (US); Adam Greenbaum, Andover, MA (US); Nasser Rafiee, Andover, MA (US); Stuart MacDonald, andover, MA (US); Mai Le Diep, andover, MA (US); Rogers Toby, andover, MA (US); Jaffar Khan, Andover, MA (US); John Horrigan, Andover, MA (US)

(21) Appl. No.: 17/038,095

(22) Filed: Sep. 30, 2020

Related U.S. Application Data
(63) Continuation of application No. PCT/US19/25383, filed on Apr. 2, 2019.
(60) Provisional application No. 62/651,692, filed on Apr. 2, 2018.

(51) Int. Cl. A61M 25/09; A61M 25/01; A61F 2/24

(52) U.S. Cl. CPC A61M 25/09; A61M 25/0147; A61M 2025/09083; A61F 2/2427; A61M 25/0152

(57) ABSTRACT

In accordance with some implementations, various embodiments of a guidewire or catheter having an elongate core with a helical section are provided. In accordance with one implementation, a guidewire is provided. The guidewire includes a core wire having a proximal end, a distal end and is defined by an outer surface between the proximal end and the distal end of the core wire. The core wire has a centerline that traverses the length of the core wire from the proximal end to the distal end of the core wire. The centerline is located approximately at the center of mass along the core wire within the outer surface of the core wire. Preferably, the core wire has a substantially straight proximal portion and a helically curved segment located distally with respect to the proximal portion. The centerline of the core wire is substantially straight along the straight proximal portion, and, the centerline is helically curved along the helically curved segment. The disclosure similarly provides a catheter having the aforementioned attributes.

### CROSS-REFERENCE TO RELATED APPLICATION

The present patent application is a continuation of and claims the benefit of priority to International Patent Application No. PCT/US2019/025383, filed Apr. 2, 2019, which in turn claims the benefit of priority to U.S. Patent Application Ser. No. 62/651,692 filed Apr. 2, 2018. The aforementioned patent application is hereby incorporated by reference in its entirety for any purpose whatsoever.

### FIELD OF THE DISCLOSURE

The present disclosure relates to various embodiments of guidewires and catheters.

### BACKGROUND

Various embodiments of guidewires and catheters are known in the art. Some of these are steerable devices. The present disclosure improves on the state of the art.

### SUMMARY OF THE DISCLOSURE

The purpose and advantages of the present disclosure will be set forth in and become apparent from the description that follows. Additional advantages of the disclosed embodiments will be realized and attained by the methods and systems particularly pointed out in the written description hereof, as well as from the appended drawings.

In accordance with some implementations, various embodiments of a guidewire or catheter having an elongate core with a helical section are provided. In accordance with one implementation, a guidewire is provided. The guidewire includes a core wire having a proximal end, a distal end and is defined by an outer surface between the proximal end and the distal end of the core wire. The core wire has a centerline that traverses the length of the core wire from the proximal end to the distal end of the core wire. The centerline is located approximately at the center of mass along the core wire within the outer surface of the core wire. Preferably, the core wire has a substantially straight proximal portion and a helically curved segment located distally with respect to the proximal portion. The centerline of the core wire is substantially straight along the straight proximal portion, and, the centerline is helically curved along the helically curved segment. The disclosure similarly provides a catheter having the aforementioned attributes. Such a catheter, if desired, can be provided with a guidewire channel along at least a part of its length.

In some implementations, a guidewire as set forth above is provided that further includes an outer tubular sleeve slidably disposed over the core wire. The outer tubular sleeve is preferably configured and arranged to be slidably disposed over the helical segment of the core wire. Slidably disposing the outer tubular sleeve over the helical segment of the core wire can cause the helicity of the helical segment of the core wire to change. For example, slidably disposing the outer tubular sleeve over the helical segment of the core wire can cause the core wire to lose helicity. If desired, slidably disposing the outer tubular sleeve over the helical segment of the core wire can cause the core wire to straighten. If desired, the helically curved segment can be pre-formed into the core wire, in the case of a guidewire, or into an elongate core or body, in the case of a catheter.

If desired, the guidewire core wire/elongate catheter core can be formed at least in part from a nickel-titanium shape memory material. The helically curved segment can be pre-formed into the core wire by a heat setting process. The helically curved segment, if desired, can be formed from a hypotube. A distal end region of the guidewire can be helically shaped, for example, to act as a spring or shock absorber when urged against tissue, such as cardiac tissue.

In some implementations, the distal end region of the guidewire or catheter can have a lower durometer, resulting in a lower stiffness, than a proximal portion of the guidewire to permit the helically shaped distal end region of the guidewire to collapse axially when urged against tissue to help absorb impact forces.

The helically curved section of the guidewire (or catheter, as appropriate) can act as a steering region. This can be accomplished, for example, by providing a gently varying helicity, such helicity having a relatively long pitch, as compared with the helical segment at the distal end of the guidewire (or catheter). By varying the axial position of the steering section of the guidewire/catheter at a target location within a patient's vasculature, the lateral position of a prosthesis disposed on a delivery catheter routed over the guidewire can be adjusted laterally to achieve improved alignment with the target location to ensure proper seating of the prosthesis (e.g., stent, stent-valve, etc.). Preferably, the steering region of the guidewire is formed at least in part from a hypotube. The hypotube can be provided with varying stiffness along its length, for example, by laser cutting grooves having various shapes into the hypotube.

The disclosure further provides a delivery system for delivering a cardiovascular prosthesis. The system includes a prosthesis disposed on a delivery catheter having a guidewire as described herein directed therethrough. If desired, the delivery catheter can be provided with controllable and adjustable helicity as described herein. The cardiovascular prosthesis can include, for example, a replacement heart valve, such as a replacement aortic, mitral or tricuspid valve.

In some implementations, the outer tubular sleeve mentioned above can be provided with a preformed curvature such as by bending or heat setting. The preformed curvature of the outer tubular sleeve can be configured so that it is geometrically opposite to the curvature of helically curved segment of the core wire, such that when the two sections are overlapping and properly aligned, the applied forces of both components cause the guidewire (or catheter, as appropriate) to be substantially or fully straight.

In various embodiments herein, the disclosed guide wires can be provided with additional components found on other known guidewires, such as one or more nested coils surrounding the core wire, atraumatic distal ends, safety wires, and the like. Examples of such features can be found in one or more of U.S. Pat. Nos. 4,827,941, 5,617,875, 4,917,103, 4,922,923, 5,031,636 and U.S. Reissue Pat. No. 34,466. Each of these patents is incorporated by reference herein in its entirety.

As alluded to above, the disclosure provides embodiments of a catheter that can include an elongate core that in turn includes a proximal end, a distal end and that is defined by an outer surface between the proximal end and the distal end of the elongate core. The elongate core has a centerline that traverses the length of the elongate core from the proximal end to the distal end of the elongate core. The centerline is located approximately at the center of mass along the elongate core within the outer surface of the elongate core. The elongate core has a substantially straight proximal portion and a helically curved segment located distally with respect to the proximal portion. The centerline of the elongate core can be substantially straight along the straight proximal portion. The centerline is typically helically curved along the helically curved segment. The catheter, if desired, can further include an outer tubular sleeve slidably disposed over the elongate core. The outer tubular sleeve can be configured and arranged to be slidably disposed over the helical segment of the elongate core. Slidably disposing the outer tubular sleeve over the helical segment of the elongate core can cause the helicity of the helical segment of the elongate core to change, such as to straighten, or to alter the curvature in other ways, such as to lose helicity. In some embodiments, the helically curved segment is pre-formed into the elongate core. The elongate core can be formed at least in part from a nickel-titanium shape memory material. The helically curved segment, made from such a shape memory material, can be pre-formed into a desired curvature by a heat setting process. If desired, the elongate core can otherwise be tubular and define a guidewire channel along at least a part of its length.

The disclosure further provides methods of delivering a prosthesis using a guidewire, catheter, or system as discussed herein that includes advancing a prosthesis into the region of a native heart valve, wherein the guidewire or catheter in the region of the native heart valve is at least partially helical, and adjusting the longitudinal or rotational position of the at least partially helical guidewire or catheter to adjust alignment of the prosthesis within the region of the native heart valve, and deploying the prosthesis into the region of the native heart valve.

In other implementations, embodiments of a guidewire (or catheter) are provided that include a tensionable tether that can alter the shape of the guidewire. For example, a guidewire can be provided that includes a core wire having a proximal end, and a distal end, at least one tubular member wrapped at least partially around the core wire, and a tensionable tether disposed within a length of the at least one tubular member. Applying tension to the tensionable tether can cause a desired (and adjustable) curvature to be imparted to the core wire.

The curvature can be generally sinusoidal. Alternatively, the at least one tubular member can be wrapped around the core wire in a generally helical pattern. The curvature can also be generally helical. In some embodiments, the at least one tubular member can be attached to the core wire. For example, the at least one tubular member can be attached to the core wire at a plurality of discrete locations along the length of the tubular member. In other embodiments, the at least one tubular member can be attached to the core wire continuously along the length of the tubular member. The at least one tubular member can be directly attached to the core wire, or attached to an intermediate structure, such as a polymeric sleeve or coating formed over the core wire. If desired, the at least one tubular member can be attached to a tubular coil disposed about the core wire. For example, the at least one tubular member can be disposed between the core wire and the tubular coiled member.

In other implementations, the at least one tubular member can include a plurality of tubular members disposed along a length of the guidewire. The tensionable tether can be disposed through at least one of the plurality of tubular members. For example, the tubular members can be disposed along the core wire in a pattern that resembles a helix. If desired, a second tensionable tether can be disposed within a length of the second tubular member, wherein applying tension to the second tensionable tether causes a curvature to be imparted to the core wire.

In some embodiments, the tensionable tether can be attached to the core wire proximate a distal end of the core wire. The tensionable tether can be attached to the core wire at the distal end of the core wire. In some embodiments, the tensionable tether can be attached to the core wire at a location that is proximal to the distal end of the core wire.

The guidewire (or catheter, as appropriate) can further include an actuator located in a proximal region of the guidewire. The actuator is typically operably coupled to the tensionable tether. Applying a tensile force to the actuator applies tension to the tensionable tether causing the guidewire to deform into a helical shape, for example.

Some implementations of a guidewire or a catheter are provided having an alternative structure for housing the tether. For example, in some embodiments, a guidewire (or catheter) is provided that includes an elongate core having a proximal end, and a distal end, at least one strip of material wrapped helically about and attached to an exterior surface of the core, wherein windings of the strip of material are spaced apart from each other to define a helical groove, an outer tubular member disposed about the at least one strip and the elongate core, wherein an outer surface of the elongate core, the helical groove formed by the strip of material, and an inner surface of the outer tubular member define a helical channel between the elongate core and the outer tubular member, and a tensionable tether disposed within the helical channel affixed to a distal region of the guidewire and extending proximally toward a proximal end of the guidewire or catheter, wherein applying tension to the tensionable tether causes a curvature to be imparted to the catheter or guidewire. If desired, the curvature imparted to the catheter or guidewire can be helical in shape.

In other implementations, a guidewire or catheter can be provided having bumps or bosses, or other structures, such as hooks formed along the surface of the core to define a helical path for the tether. For example, a guidewire or catheter can be provided that includes an elongate core having a proximal end, and a distal end, a plurality of bosses or protrusions extending radially outwardly from an exterior surface of the elongate core, wherein the bosses or protrusions form a helical pattern around the exterior surface of the elongate core, an outer tubular member disposed about the elongate core, wherein the plurality of bosses are urged against an inner surface of the outer tubular member, and a tensionable tether disposed between the elongate core and the outer tubular member, wherein the tether is affixed to a distal region of the guidewire or catheter. The tether is wound helically around the elongate core underneath the outer tubular member toward a proximal end of the guidewire or catheter, wherein applying tension to the tensionable tether causes the tether to pull against the bosses or protrusions and impart a curvature to the guidewire or catheter. If desired, the curvature imparted to the catheter or guidewire can be helical in shape or may lay in a two dimensional plane. The outer tubular member can be fused to the plurality of bosses or protrusions such that the elongate core and outer tubular member are an integral unit.

In other implementations, guidewires or catheters are provided having an inner elongate member having a helical groove formed into its outer surface, with an outer tube disposed over the groove to form a helical channel for routing a tether (or pull wire), as described herein. For example such a guidewire or catheter can include an elongate core having a proximal end, and a distal end, the elongate core having a helical groove formed along an exterior surface thereof along the length of the elongate core, an outer tubular member disposed about the elongate core, wherein an outer surface of the elongate core, the helical groove, and an inner surface of the outer tubular member define a helical channel between the elongate core and the outer tubular member, and a tensionable tether disposed within the helical channel affixed to a distal region of the guidewire or catheter and extending proximally toward a proximal end of the guidewire or catheter, wherein applying tension to the tensionable tether causes a curvature to be imparted to the guidewire or catheter. The curvature imparted to the catheter or guidewire can be helical in shape, for example.

Embodiments of catheters and guidewires are also provide having a helical core section with a further tube helically wound around the helical core section with a tensionable tether in it. For example, a guidewire or catheter can be provided including a core wire having a proximal end, and a distal end and being defined by an outer surface between the proximal end and the distal end of the core wire, the core wire having a centerline that traverses the length of the core wire from the proximal end to the distal end of the core wire, the centerline being located approximately at the center of mass along the core wire within the outer surface of the core wire, the core wire having a substantially straight proximal portion and a helically curved segment located distally with respect to the proximal portion, wherein the centerline of the core wire is substantially straight along the straight proximal portion and further wherein the centerline is helically curved along the helically curved segment. The guidewire or catheter can further include at least one tubular member helically wound around the helical segment of the core wire, and a tensionable tether disposed within the tubular member, wherein applying tension to the tensionable tether causes the guidewire or catheter to change in shape.

If desired, implementations of guidewires and catheters are provided having a push rod disposed therein. The push rod can be movable within a tubular member that is wound at least partially around a core member of the guidewire or catheter. Thus, in some implementations, a guidewire or catheter is provided that includes an elongate core having a proximal end, and a distal end and defined by an outer surface between the proximal end and the distal end of the elongate core. The elongate core has a centerline that traverses the length of the elongate core from the proximal end to the distal end of the elongate core, the centerline being located approximately at the center of mass along the elongate core within the outer surface of the elongate core. The elongate core has a substantially straight proximal portion and a helically curved segment located distally with respect to the proximal portion, wherein the centerline of the elongate core is substantially straight along the straight proximal portion and further wherein the centerline is helically curved along the helically curved segment. The catheter or guidewire further includes at least one (or two or three or more) tubular member(s) helically wound around the helical segment of the elongate core, and a push rod disposed within the at least one tubular member, wherein applying compression to the push rod causes the helically curved segment to straighten. If additional tubular members are provided, they can each be provided with their own pushrod or wire to adjust the curvature of the catheter or guidewire, as desired.

In another implementation, a guidewire or catheter is provided that can be deformed into a helical shape. The guidewire or catheter can include an elongate core having a proximal end, and a distal end and can be defined by an outer surface between the proximal end and the distal end of the elongate core. The elongate core can have a centerline that traverses the length of the elongate core from the proximal end to the distal end of the elongate core. The centerline can be located approximately at the center of mass along the elongate core within the outer surface of the elongate core. A tubular member can be helically wound around the helical segment of the elongate core. A push rod can be disposed within the tubular member. Applying compression to the push rod causes the guidewire or catheter to take on a helical curvature.

It is to be understood that both the foregoing general description and the following detailed description are exemplary and are intended to provide further explanation of the embodiments disclosed herein.

The accompanying drawings, which are incorporated in and constitute part of this specification, are included to illustrate and provide a further understanding of the method and system of the disclosure. Together with the description, the drawings serve to explain the principles of the disclosed embodiments.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings, in which:

FIG. 1 depicts a first representative implementation of a guidewire or catheter in accordance with the present disclosure.

FIGS. 2A-2B illustrate a second representative embodiment of a guidewire or catheter in accordance with the present disclosure.

FIGS. 2C-2E illustrate aspects of a third representative embodiment of a guidewire or catheter in accordance with the present disclosure.

FIGS. 3A-3C illustrate aspects of a fourth representative embodiment of a guidewire or catheter in accordance with the present disclosure.

FIG. 4 illustrates aspects of a medical procedure using a guidewire or catheter in accordance with the present disclosure.

FIGS. 5A-5B illustrate further aspects of a medical procedure using a guidewire or catheter in accordance with the present disclosure.

### DETAILED DESCRIPTION

Reference will now be made in detail to the present preferred embodiments of the disclosure, examples of which are illustrated in the accompanying drawings. The method and corresponding steps of the disclosed embodiments will be described in conjunction with the detailed description of the system. The exemplary embodiments illustrated herein can be used to perform various procedures, but percutaneously. It will be appreciated, however, that the disclosed embodiments, or variations thereof, can be used for a multitude of procedures involving the connection of blood vessels or other biological lumens to native or artificial structures.

In accordance with some implementations, various embodiments of a guidewire or catheter having an elongate core with a helical section are provided. In accordance with one implementation, a guidewire is provided.

For purposes of illustration, and not limitation, as embodied herein and as illustrated in FIG. 1, a guidewire is provided that includes a core wire 100 having a proximal end 102, a distal end 104 and is defined by an outer surface between the proximal end and the distal end of the core wire. The core wire has a centerline C that traverses the length of the core wire 100 from the proximal end to the distal end of the core wire and coincides with a longitudinal axis X of the device in certain locations. In other locations, the centerline spirals around the axis X along a helical path having a radius R, and inner and outer radii n and r₀. The centerline C is located approximately at the center of mass along the core wire 100 within the outer surface of the core wire 100. That is to say, the centerline is intended to describe how the core wire spirals around the axis X. Preferably, the core wire 100 has a substantially straight proximal portion 110 and a helically curved segment 120 located distally with respect to the proximal portion. A flexible distal portion 130 is also provided having a softer spring-like distal coil. The centerline C of the core wire 100 is substantially straight along the straight proximal portion 110, and, the centerline C is helically curved along the helically curved segment 120, as well as the distal segment 130. Those of skill in the art will recognize that, while a core wire 100 of a guidewire is illustrated, these teachings and geometry can just as easily be used to create a catheter, such as (but not limited to) a catheter for delivering a prosthesis. The radius R can range from, for example, the diameter of the core wire up to about 5× the diameter of the core wire, in increments of about 0.1× of the diameter of the core wire, inclusive of the end points of said range. The core wire 100 can have an outer diameter, for example, between about 0.012 and about 0.040 inches, inclusive of the endpoints of said range, and any increment therebetween of about 0.001 inches. As further illustrated in FIG. 1, the core wire 100 includes a pitch P that represents the distance over which the helix repeats. The pitch of guidewires and catheters herein can be between, for example, 5 mm and about 100 mm, inclusive of said endpoints, in increments of about 1.0 mm.

In some implementations, a guidewire as set forth above is provided that further includes an outer tubular sleeve slidably disposed over the core wire. The outer tubular sleeve is preferably configured and arranged to be slidably disposed over the helical segment of the core wire. Slidably disposing the outer tubular sleeve over the helical segment of the core wire can cause the helicity of the helical segment of the core wire to change.

For purposes of illustration, and not limitation, as illustrated in FIGS. 2A-2B, a guidewire is provided having a core wire 100 similar to that presented in FIG. 1, and an outer sleeve 200 that is slidably disposed over the core wire 100, and if desired, connected to core wire 100 via actuator A. Any desired actuator A can be used, such as threaded rotating actuators as described in U.S. Pat. No. 6,488,694 to Lau and U.S. Pat. No. 5,906,619 to Olson, each of which is incorporated by reference herein in its entirety. Such actuators can be used, as appropriate, for any embodiment of a guidewire or catheter herein.

As illustrated, the embodiment has a sleeve 200 that is substantially straight and somewhat rigid, so that when it is advanced distally with respect to the core wire and covers the helical region 120 of the core wire, some or all of the helicity and curvature is lost. As illustrated, helically curved segment 120 of the core wire 100 is pre-formed into the core wire 100. This can also be implemented with respect to the elongate core or body of a catheter (not illustrated).

If desired, the guidewire core wire/elongate catheter core can be formed at least in part from a nickel-titanium shape memory material (e.g., Nitinol® material). The helically curved segment can be pre-formed into the core wire by a heat setting process. The helically curved segment, if desired, can be formed from a hypotube. A distal end region of the guidewire can be helically shaped, for example, to act as a spring or shock absorber when urged against tissue, such as cardiac tissue.

In some implementations, the distal end region of the guidewire or catheter can have a lower durometer, resulting in a lower stiffness, than a proximal portion of the guidewire to permit the helically shaped distal end region of the guidewire to collapse axially when urged against tissue to help absorb impact forces. Portions of the guidewires or catheters disclosed herein may be made in a variety of ways and from a variety of materials, such as metal, plastic and composite materials. Metal tubes such as stainless steel hypotubes can be used for a proximal portion, 110, for example, of the core wire 100. Carbon tubes and the like can be used, or polymeric tubes formed, for example, from polyester, PEEK, Nylon® materials and the like. If metal components are used to make portions of catheters or guidewires disclosed herein, they are preferably coated with a lubricious material such as PTFE, PVDF other hydrophobic materials or hydrophilic materials such as PVP.

Multilayered polymeric tubes can also be used that include metallic or nonmetallic braiding within or between layers of the tube. Carbon tubes can also be used, as well as fiber-reinforced resin materials. Multilayered polymeric tubes can also be used to form portions of the guidewires or catheters set forth herein that can be formed, for example, by co-extrusion, dipping processes, or by shrinking tubing layers over one another over a mandrel. For example, such polymeric tubes can include a multi-layered co-extrusion, such as those described in U.S. Pat. No. 6,464,683 to Samuelson or U.S. Pat. No. 5,538,510 to Fontirroche. Each of the aforementioned patents is incorporated by reference herein in its entirety.

Moreover, polymeric tubular members can also be formed by charging a mandrel with static electricity, applying plastic in powder or granular form to the mandrel to form a layer of plastic over the mandrel, and by heating the mandrel to cause the particles to fuse. If desired, stiffening members such as stiffening wires or carbon rods can be used at various locations along portions of the disclosed guidewires or catheters to provide stiffness transitions between relatively stiffer regions and less stiff regions, as well as in regions of stress concentration.

In some implementations, the outer tubular sleeve 200 can be provided with a preformed curvature such as by bending or heat setting. The preformed curvature of the outer tubular sleeve can be configured so that it is geometrically opposite to the curvature of helically curved segment of the core wire, such that when the two sections are overlapping and properly aligned, the applied forces of both components cause the guidewire (or catheter, as appropriate) to be substantially or fully straight.

In various embodiments herein, the disclosed guide wires can be provided with additional components found on other known guidewires, such as one or more nested coils surrounding the core wire, atraumatic distal ends, safety wires, and the like. Examples of such features can be found in one or more of U.S. Pat. Nos. 4,827,941, 5,617,875, 4,917,103, 4,922,923, 5,031,636 and U.S. Reissue Pat. No. 34,466. Each of these patents is incorporated by reference herein in its entirety.

In other implementations, embodiments of a guidewire (or catheter) are provided that include a tensionable tether that can alter the shape of the guidewire.

For purposes of illustration, and not limitation, as illustrated in FIGS. 3A-3C, a guidewire 300 is provided that includes a core wire 310 having a proximal end 312, and a distal end 314. The guidewire 300 further includes, at least one tubular member 320 wrapped at least partially around the core wire 310. Further, a tensionable tether 330 can be disposed along and within the lumen of the at least one tubular member 320. Applying proximally directed tension to the tensionable tether 330 can cause a desired (and adjustable) curvature to be imparted to the core wire 310. The curvature can be generally sinusoidal (e.g., in a two dimensional plane). The curvature can also be generally helical, as illustrated.

Accordingly, the at least one tubular member 320 can be wrapped around the core wire in a generally helical pattern as illustrated in FIG. 3A. In some embodiments, the at least one tubular member 320 can be attached to the core wire 310. For example, the at least one tubular member 320 can be attached to the core wire 310 at a plurality of discrete locations along the length of the tubular member 320. In other embodiments, the at least one tubular member 320 can be attached to the core wire 310 continuously along the length of the tubular member 320. The at least one tubular member 310 can be directly attached to the core wire, or attached to an intermediate structure, such as a polymeric sleeve or coating formed over the core wire 310. If desired, the at least one tubular member can be attached to a tubular coil (not shown) disposed about the core wire. For example, the at least one tubular member can be disposed between the core wire and the tubular coiled member.

In other implementations that are not specifically illustrated, the at least one tubular member can include a plurality (e.g., 2, 3) tubular members that are wrapped parallel to each other around the core wire, thereby forming a plurality of parallel helical passages that surround the core wire 310. A tensionable tether can be provided in each of the helically wound tubular members 320, having distal ends 334 that terminate at different locations disposed along the core wire in order to cause different portions of the core wire to deform. In some embodiments, the tensionable tether(s) can be attached to the core wire proximate a distal end of the core wire or in multiple locations near the distal end of the core wire. In some embodiments, one or more of the tensionable tether(s) can be attached to the core wire at a location that is proximal to the distal end of the core wire.

This embodiment of the guidewire (or catheter, as appropriate) can further include an actuator A located in a proximal region of the guidewire. The actuator is typically operably coupled to the tensionable tether 330. Applying a tensile force to the actuator applies tension to the tensionable tether causing the guidewire 300 to deform into a helical shape, for example. Any of the actuators set forth in can be used to tension the tether, for example.

Some implementations of a guidewire or a catheter are provided having an alternative structure for housing the tether. For example, in some embodiments, a guidewire (or catheter) is provided that includes an elongate core having a proximal end, and a distal end, at least one strip of material wrapped helically about and attached to an exterior surface of the core (not specifically illustrated), wherein windings of the strip of material are spaced apart from each other to define a helical groove, an outer tubular member disposed about the at least one strip and the elongate core, wherein an outer surface of the elongate core, the helical groove formed by the strip of material, and an inner surface of the outer tubular member define a helical channel between the elongate core and the outer tubular member, and a tensionable tether disposed within the helical channel affixed to a distal region of the guidewire and extending proximally toward a proximal end of the guidewire or catheter, wherein applying tension to the tensionable tether causes a curvature to be imparted to the catheter or guidewire. If desired, the curvature imparted to the catheter or guidewire can be helical in shape.

In other implementations, a guidewire or catheter can be provided (not specifically illustrated in the Figures) having bumps or bosses, or other structures, such as hooks formed along the surface of the core to define a helical path for the tether. For example, a guidewire or catheter can be provided that includes an elongate core having a proximal end, and a distal end, a plurality of bosses or protrusions extending radially outwardly from an exterior surface of the elongate core, wherein the bosses or protrusions form a helical pattern around the exterior surface of the elongate core, an outer tubular member disposed about the elongate core, wherein the plurality of bosses are urged against an inner surface of the outer tubular member, and a tensionable tether disposed between the elongate core and the outer tubular member, wherein the tether is affixed to a distal region of the guidewire or catheter. The tether is wound helically around the elongate core underneath the outer tubular member toward a proximal end of the guidewire or catheter, wherein applying tension to the tensionable tether causes the tether to pull against the bosses or protrusions and impart a curvature to the guidewire or catheter. If desired, the curvature imparted to the catheter or guidewire can be helical in shape or may lay in a two dimensional plane. The outer tubular member can be fused to the plurality of bosses or protrusions such that the elongate core and outer tubular member are an integral unit.

In other implementations, guidewires or catheters are provided (not specifically illustrated in the Figures) having an inner elongate member having a helical groove formed into its outer surface, with an outer tube disposed over the groove to form a helical channel for routing a tether (or pull wire), as described herein. For example such a guidewire or catheter can include an elongate core having a proximal end, and a distal end, the elongate core having a helical groove formed along an exterior surface thereof along the length of the elongate core, an outer tubular member disposed about the elongate core, wherein an outer surface of the elongate core, the helical groove, and an inner surface of the outer tubular member define a helical channel between the elongate core and the outer tubular member, and a tensionable tether disposed within the helical channel affixed to a distal region of the guidewire or catheter and extending proximally toward a proximal end of the guidewire or catheter, wherein applying tension to the tensionable tether causes a curvature to be imparted to the guidewire or catheter. The curvature imparted to the catheter or guidewire can be helical in shape, for example.

As illustrated in FIGS. 4, 5A and 5B, the helically curved section 120 of the guidewire/core wire 100 (or catheter, as appropriate) can act as a steering region. This can be accomplished, for example, by providing a gently varying helicity, such helicity having a relatively long pitch, as compared with the helical segment at the distal end of the guidewire (or catheter). As illustrated in FIGS. 5A-5B, by varying the axial position of the steering section 120 of the guidewire/catheter at a target location within a patient's vasculature, the lateral (i.e., side-to-side) position of a prosthesis, such as a replacement aortic valve, disposed on a delivery catheter routed over the guidewire can be adjusted laterally to achieve improved alignment with the target location to ensure proper seating of the prosthesis (e.g., stent, stent-valve, etc.). This helps compensate for the fact that guidewires tend to track along the outer radius of curved vessels into which they are inserted. For example, proximal section no in FIG. 4 is tracking along the outer radius of the aorta. By providing a helical section of guidewire that can be adjusted in helicity, as well as axial location, it becomes possible to more fully align a delivery system and replacement valve in the native location of the original aortic valve. Preferably, the steering region of the guidewire is formed at least in part from a hypotube, but can be formed from alternative materials as set forth herein. The hypotube can be provided with varying stiffness along its length, for example, by laser cutting grooves having various shapes into the hypotube. Also, if the hypotube forming the steering region 120 is formed from shape memory material, it can be heat set to deploy once unsheathed, for example, by a tubular member 200 or retractable sheath or microcatheter in which core wire 100 is disposed.

Embodiments of catheters and guidewires are also provided (not specifically illustrated in the Figures) having a helical core section with a further tube helically wound around the helical core section with a tensionable tether in it. For example, a guidewire or catheter can be provided including a core wire having a proximal end, and a distal end and being defined by an outer surface between the proximal end and the distal end of the core wire, the core wire having a centerline that traverses the length of the core wire from the proximal end to the distal end of the core wire, the centerline being located approximately at the center of mass along the core wire within the outer surface of the core wire, the core wire having a substantially straight proximal portion and a helically curved segment located distally with respect to the proximal portion, wherein the centerline of the core wire is substantially straight along the straight proximal portion and further wherein the centerline is helically curved along the helically curved segment. The guidewire or catheter can further include at least one tubular member helically wound around the helical segment of the core wire, and a tensionable tether disposed within the tubular member, wherein applying tension to the tensionable tether causes the guidewire or catheter to change in shape.

If desired, implementations of guidewires and catheters are provided herein (not specifically illustrated in the Figures) having a push rod disposed therein. The push rod can be movable within a tubular member that is wound at least partially around a core member of the guidewire or catheter. Thus, in some implementations, a guidewire or catheter is provided that includes an elongate core having a proximal end, and a distal end and defined by an outer surface between the proximal end and the distal end of the elongate core. The elongate core has a centerline that traverses the length of the elongate core from the proximal end to the distal end of the elongate core, the centerline being located approximately at the center of mass along the elongate core within the outer surface of the elongate core. The elongate core has a substantially straight proximal portion and a helically curved segment located distally with respect to the proximal portion, wherein the centerline of the elongate core is substantially straight along the straight proximal portion and further wherein the centerline is helically curved along the helically curved segment. The catheter or guidewire further includes at least one (or two or three or more) tubular member(s) helically wound around the helical segment of the elongate core, and a push rod disposed within the at least one tubular member, wherein applying compression to the push rod causes the helically curved segment to straighten. If additional tubular members are provided, they can each be provided with their own pushrod or wire to adjust the curvature of the catheter or guidewire, as desired.

In another implementation, a guidewire or catheter is provided (not specifically illustrated in the Figures) that can be deformed into a helical shape. The guidewire or catheter can include an elongate core having a proximal end, and a distal end and can be defined by an outer surface between the proximal end and the distal end of the elongate core. The elongate core can have a centerline that traverses the length of the elongate core from the proximal end to the distal end of the elongate core. The centerline can be located approximately at the center of mass along the elongate core within the outer surface of the elongate core. A tubular member can be helically wound around the helical segment of the elongate core. A push rod can be disposed within the tubular member. Applying compression to the push rod causes the guidewire or catheter to take on a helical curvature.

The devices and methods disclosed herein can be used for other procedures in an as-is condition, or can be modified as needed to suit the particular procedure. In view of the many possible embodiments to which the principles of this disclosure may be applied, it should be recognized that the illustrated embodiments are only preferred examples of the disclosure and should not be taken as limiting the scope of the disclosure.

What is claim is:

1. A guidewire, comprising a core wire having a proximal end, and a distal end and being defined by an outer surface between the proximal end and the distal end of the core wire, said core wire having a centerline that traverses the length of the core wire from the proximal end to the distal end of the core wire, said centerline being located approximately at the center of mass along said core wire within said outer surface of said core wire, said core wire having a substantially straight proximal portion and a helically curved segment located distally with respect to said proximal portion, wherein:
the centerline of the core wire is substantially straight along said straight proximal portion;
said centerline is helically curved along said helically curved segment;
a distal end region of the guidewire is helically shaped; and
the distal end region has a lower durometer than a proximal portion of the guidewire to permit the helically shaped distal end region of the guidewire to collapse axially when urged against tissue.

2. The guidewire of claim 1, further comprising an outer tubular sleeve slidably disposed over said core wire wherein said outer tubular sleeve is configured and arranged to be slidably disposed over said helical segment of said core wire, and slidably disposing said outer tubular sleeve over said helical segment of said core wire causes the helicity of said helical segment of said core wire to change.

3. The guidewire of claim 2, wherein slidably disposing said outer tubular sleeve over said helical segment of said core wire causes said core wire to lose helicity.

4. The guidewire of claim 2, wherein slidably disposing said outer tubular sleeve over said helical segment of said core wire causes said core wire to straighten.

5. The guidewire of claim 1, wherein the helically curved segment is pre-formed into the core wire.

6. The guidewire of claim 2, wherein said outer tubular sleeve has a preformed curvature.

7. The guidewire of claim 6, wherein the preformed curvature of the outer tubular sleeve is geometrically opposite to the curvature of helically curved segment of the core wire, and further wherein the guidewire is substantially straight when the outer tubular sleeve overlays the helically curved segment of the core wire.

8. A delivery system for a cardiovascular prosthesis comprising a prosthesis disposed on a delivery catheter, and a guidewire disposed through at least a portion of the length of the delivery catheter, the guidewire including a core wire having a proximal end, and a distal end and being defined by an outer surface between the proximal end and the distal end of the core wire, said core wire having a centerline that traverses the length of the core wire from the proximal end to the distal end of the core wire, said centerline being located approximately at the center of mass along said core wire within said outer surface of said core wire, said core wire having a substantially straight proximal portion and a helically curved segment located distally with respect to said proximal portion, wherein:
the centerline of the core wire is substantially straight along said straight proximal portion; and
said centerline is helically curved along said helically curved segment.

9. The delivery system of claim 8, wherein the cardiovascular prosthesis includes a replacement heart valve.

10. The guide wire or delivery system of claim 1, wherein at least a portion of the core wire is surrounded by a coil that is attached to the core wire in at least one location.

11. A method of delivering a prosthesis using a guidewire according to claim 1, comprising advancing a prosthesis into the region of a native heart valve, wherein the guidewire in the region of the native heart valve is at least partially helical, and adjusting the longitudinal or rotational position of the at least partially helical guidewire to adjust alignment of the prosthesis within the region of the native heart valve, and deploying the prosthesis into the region of the native heart valve.

12. A guidewire, comprising:
a core wire having a proximal end, and a distal end;
at least one tubular member wrapped at least partially around said core wire; and
a tensionable tether disposed within a length of said at least one tubular member, wherein applying tension to said tensionable tether causes a curvature to be imparted to said core wire.

13. The guidewire of claim 12, wherein said curvature is generally sinusoidal.

14. The guidewire of claim 12, wherein said at least one tubular member is wrapped around said core wire in a generally helical pattern.

15. The guidewire of claim 14, wherein said curvature is generally helical.

16. The guidewire of claim 12, wherein said at least one tubular member is attached to said core wire.

17. The guidewire of claim 16, wherein said at least one tubular member is attached to said core wire at a plurality of discrete locations along the length of said tubular member.

18. The guidewire of claim 16, wherein said at least one tubular member is attached to said core wire continuously along the length of said tubular member.

19. The guidewire of claim 12, wherein said at least one tubular member is directly attached to said core wire.

20. The guidewire of claim 12, wherein said at least one tubular member is attached to a tubular coil disposed about said core wire.
